(19) United States
(12) Patent Application Publication
(10) Pub. No.: US 2015/0297191 A1
(43) Pub. Date: Oct. 22, 2015

(54) Ultrasound Transducer

(71) Applicant: SOUND TECHNOLOGY INC., State College, PA (US)

(72) Inventor: Christopher Beers, Boalsburg, PA (US)

(21) Appl. No.: 14/647,927

(22) PCT Filed: Nov. 29, 2012
(86) PCT No.: PCT/US2012/066970

§ 371 Date: May 28, 2015

(51) Int. Cl. A61B 8/00

(52) U.S. Cl. CPC A61B 8/54; A61B 8/4494; A61B 8/465

(57) ABSTRACT

An imaging probe includes a transducer array with transducer elements (109) with parallel first and second planar surface (201, 203) in which an ultrasound signal is emitted from the first planar surface. A transducer element, includes a plurality of transducing sub-elements (208) arranged along an elevation direction in which adjacent transducing sub-elements are separated from each other by kerfs (212) of non-transducing material, wherein depths of the kerfs vary along the elevation direction.

### TECHNICAL FIELD

The following generally relates to an ultrasound transducer and is described with particular application herein to ultrasound imaging.

### BACKGROUND

Ultrasound (US) image quality is adversely affected by the spread of the acoustic energy perpendicular to the imaging plane. Energy that spreads outside of this plane degrades the image by capturing confounding features in the image, thereby reducing the overall image signal-to-noise ratio. Ideally, the energy would be narrower and collimated in the imaging plane. However, with one-dimensional arrays, some non-negligible fraction of the radiated energy spreads outside of the imaging plane.

The out-of-plane energy can be in the form of general spreading of the central on-axis energy lobe or as discrete sidelobes. The energy tends to spread increasingly at greater depths, so deep imaging applications (with a large abdominal probe, for instance) may be particularly susceptible to sidelobes. Also, when the features are very small (for example, applications that might call for a high-frequency linear probe), excess image clutter induced by out-of-plane energy is especially unhelpful.

Ultrasound image quality is also affected by the variation of the focal depth across the frequency range. Tissue attenuation is greater for higher frequencies, so the higher frequencies emitted by the array are attenuated more than the lower frequencies. Consequently, the higher frequencies are generally more useful for shallower imaging since their penetration is limited by the tissue attenuation, and the lower frequencies are more useful for deeper imaging since the low-frequency penetration is greater.

A transducer array has relatively uniform frequency response across its elevation and a natural focus, which typically is wider and deeper than desired. An acoustic lens provides a narrower focus at a depth of interest. The geometric focal depth applied by the acoustic lens is mostly independent of frequency, but the natural focal depth increases with increasing frequency. As a result, the net acoustic focal depth is shallower for lower frequencies and deeper for higher frequencies, which, unfortunately, is opposite the desired relationship between focal depth and frequency established by tissue attenuation.

### SUMMARY

Aspects of the application address the above matters, and others.

In one aspect, an imaging probe includes a transducer array, with transducer elements with parallel first and second planar surface in which an ultrasound signal is emitted from the first planar surface, a transducer element, including: a plurality of transducing sub-elements arranged along an elevation direction in which adjacent transducing sub-elements are separated from leach other by kerfs of non-transducing material, wherein depths of the kerfs vary along the elevation direction.

In another aspect, a method includes exciting a transducer array, thereby producing an ultrasound beam that traverses an examination field of view, wherein the transducer array elements include a plurality of sub-elements arranged along an elevation direction wherein each element in elevation has kerfs of non-transducing material located between sub-elements, wherein depths of the kerfs vary along the elevation direction.

In another aspect, an ultrasound imaging system includes an imaging probe with transducer array elements that include sub-elements arranged along an elevation direction in which adjacent sub-elements are separated from each other by kerfs of non-transducing material with depths that extend only part way through the sub-elements and a console in electrical communication with the imaging probe, wherein the console controls transmission of an ultrasound signal by the array and processes echoes received by the array.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example imaging probe with a transducer array in connection with an imaging console.

FIG. 2 schematically illustrates an example of a transducer element of the transducer array of FIG. 1 in which the element includes spatially varying kerf depths.

FIG. 3 shows an excitation profile of the transducer element of FIG. 2.

FIG. 4 shows a pressure profile at a focus depth for the excitation profile of FIG. 3.

FIG. 5 shows an excitation profile of a prior art transducer element with equal or no kerfs.

FIG. 6 shows a pressure profile at a focus depth for the excitation profile of FIG. 5.

FIG. 7 schematically illustrates another example of the transducer element in which the element includes spatially varying kerf depths (continuous), kerf widths, and post widths.

FIG. 8 schematically illustrates another example of the transducer element in which the element includes spatially varying kerf depths (discrete), kerf widths, and post widths.

FIG. 9 schematically illustrates the transducer element of FIG. 7 in connection with a first electrode configuration.

FIG. 10 schematically illustrates the transducer element of FIG. 7 in connection with another electrode configuration.

FIG. 11 schematically illustrates the transducer element of FIG. 7 in connection with electrodes, a backing support, and multiple layers of impedance matching layers.

FIG. 12 schematically illustrates another transducer element in connection with electrodes, a backing support, and multiple layers of impedance matching layers.

FIG. 13 illustrates an example method in accordance with an example imaging probe having varying kerf depths.

FIG. 14 illustrates an example method in accordance with an example imaging probe having varying kerf depths and varying post widths.

FIG. 15 illustrates an example method in accordance with an example imaging probe having varying kerf depths and varying kerf widths.

FIG. 16 illustrates an example method in accordance with an example imaging probe having varying kerf depths, varying kerf widths, and varying post widths.

FIG. 17 illustrates an embodiment in which a transducer element has kerf depths that increase monotonically from outer edges to a central region, symmetrically about the central region.

FIG. 18 illustrates an embodiment in which a transducer element has kerf depths that vary symmetrically about a central region, but not monotonically between outer edges and the central region.

FIG. 19 illustrates an embodiment in which a transducer element has kerf depths that vary asymmetrically about a central region, but not monotonically between outer edges and the central region.

FIG. 20 illustrates an embodiment in which a transducer element has equal kerf depths that do not extend completely through the sub-element.

FIG. 21 illustrates an embodiment in which a plurality of sub-elements are arranged to form a 1D array of sub-elements.

### DETAILED DESCRIPTION

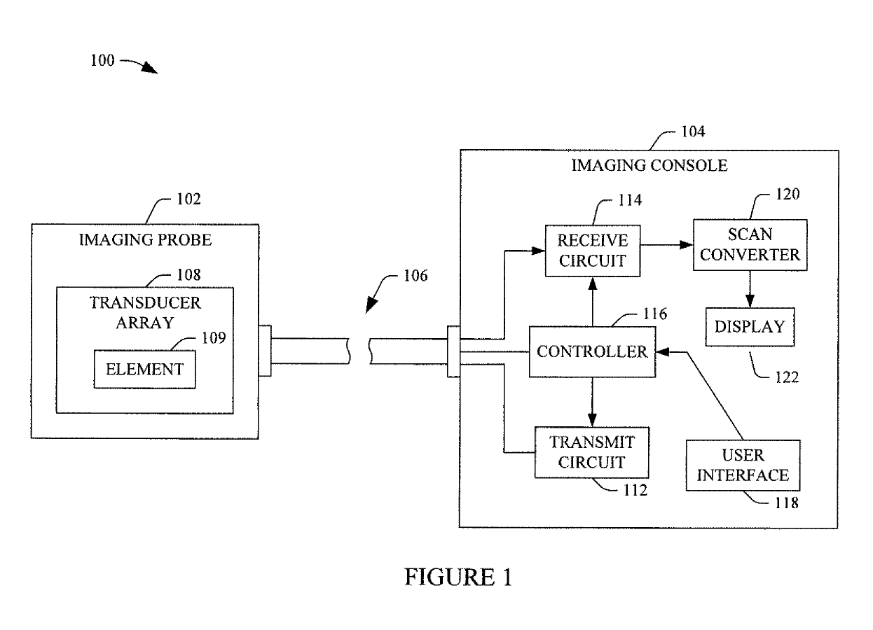

FIG. 1 illustrates a non-limiting example imaging system 100 such as an ultrasound imaging system. The imaging system 100 includes an imaging probe 102 and an imaging console 104, which are in electrical communication through a communications channel 106.

The imaging probe 102 includes a one dimensional transducer array 108 consisting of at least one transducer (e.g., piezoelectric) elements 109. As described in greater detail below, in one non-limiting instance, a shape of an element 109 in transducer array 108 is a rectangular prism or parallelepiped and includes a plurality of transducer (e.g., piezoelectric) sub-elements or posts, which are separated from each other by kerfs filled with a passive or non-transducing material. In one instance, depths of the kerfs spatially vary in size, continuously or in discrete steps, across an elevation direction, from deeper to shallower, from ends of the element 109 towards a central region of the element 109. Additionally or alternatively, widths of the kerf and/or widths of the posts likewise spatially vary in size across the elevation direction.

In one non-limiting instance, such spatial variations lead to a spatially-varying response in magnitude of the element 109. For example, regions with deeper kerfs have less vibration relative to regions with shallower kerfs. As such, the magnitude of the excitation energy rolls off nearer the ends of the element 109 relative to the central region of the element 109, thereby mitigating side lobes and improving image quality. The spatial variations also lead to a spatially-varying response in frequency of the element 109. For example, regions with deeper kerfs have a lower resonance frequency relative to regions with shallower kerfs. As such, the probe 102 is well-suited for both deep (lower frequency) and shallow (higher frequency) imaging applications.

The imaging console 104 includes a transmit circuit 112 that controls phasing and/or time of excitation of the elements of the transducer array 108, which allows for steering and/or focusing the transmitted beam from predetermined origins along the array and at predetermined angles. The ultrasound imaging console 104 also includes receive circuit 114 that receives the echoes received by the transducer array 108. For B-mode and/or other applications, the receive circuit 114 beamforms (e.g., delays and sums) the echoes from the transducer elements into a sequence of focused, coherent echo samples along focused scanlines of a scanplane. In other embodiments, the receive circuit 114 otherwise processes the echoes. Examples of other imaging techniques include, but are not limited to, synthetic aperture, shear wave elastography, etc., which may employ other computational approaches.

A controller 116 of the ultrasound imaging console 104 controls the transmit circuit 112 and/or the receive circuit 114. Such control may include, but is not limited to, controlling the frame rate, number of scanline groups, transmit angles, transmit energies, transmit frequencies, transmit and/or receive delays, the imaging mode (e.g., B-mode, C-mode, Doppler, etc.), etc. A user interface 118 includes various input and/or output devices for interacting with the controller 116, for example, to select a data acquisition mode, a data processing mode, a data presentation mode, etc. The user interface 118 may include various controls such as buttons, knobs, a keypad, a touch screen, etc. The user interface 118 may also include various types of visual and/or audible indicators.

A scan converter 120 of the ultrasound imaging console 104 scan converts the frames of data to generate data for display, for example, by converting the data to the coordinate system of the display. The scan converter 120 can be configured to employ analog and/or digital scan converting techniques. A display 122 can be used to present the acquired and/or processed data. Such presentation can be in an interactive graphical user interface (GUI), which allows the user to selectively rotate, scale, and/or manipulate the displayed data. Such interaction can be through a mouse or the like and/or a keyboard or the like. The display 122 can alternatively be remote from the console 104.

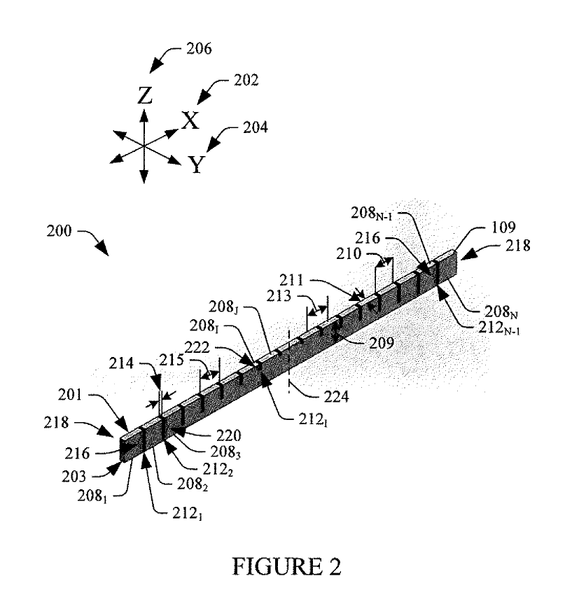

FIG. 2 illustrates a perspective view 200 of an example of the transducer element 109 in elevation, azimuth and depth directions 202, 204 and 206. The transducer element 109 is a rectangular prism with planar surfaces 201 and 203 that extend parallel to each other along the elevation direction 202, where the ultrasound beam is emitted toward the patient from the surface 201. Of course, some ultrasound energy also travels away from the patient.

The transducer element 109 includes N transducing sub-elements or posts 208₁, 208₂, 208₃, . . . , 208_{I}, 208_{J}, . . . , 208_{N-1}, 208_{N} (where N is an integer), collectively referred to herein as posts 208. In the illustrated example, a height 209 (depth direction) of the posts 208 is greater than widths 210 (elevation direction) of the posts 208, which is greater than thicknesses 211 (azimuth direction) of the posts. In this example, all of the posts 208 have the same height 209, the same width 210 and a same pitch 213 (center to center distance). In a variation, at least two posts 208 have a different height 209 and/or same width 210, and/or a same pitch 213 relative to another pair of posts 208.

The posts 208 are separated by N−1 kerfs 212₁, 212₂, . . . , 212_{I}, . . . 212_{N-1}, collectively referred to herein as kerfs 212, which include a non-transducing material. Likewise, the kerfs 212 have a same width 214 and a same pitch 215, and thickness equal to the thickness 211 of the posts 208. However, in the illustrated embodiment, depths of kerfs 212 vary along the elevation direction 202, with greater depths 216 at end regions 218 and decreasing depths 220 and 222 approaching a central region 224. In other embodiments, as discussed in greater detail below, the depths of the kerfs 212 vary along the elevation direction, with greater depths at the central region, decreasing towards the end regions, the depths of kerfs 212 vary along the elevation direction neither monotonically increasing nor monotonically decreasing, the depths of the kerfs vary along the elevation direction symmetrically or asymmetrically, etc.

In the illustrated example, the depths of kerfs 212 vary symmetrically about the central region 224. In a variation, the depths of kerfs 212 vary asymmetrically about the central region 224. Furthermore, in the illustrated example, the change in the depths of the kerf 212 from the ends 218 to the central region 224 is smooth and gradual. In a variation, the depths of kerfs 212 vary in groups in a step-wise manner. As described below, in other embodiments, the kerf width 214 and/or the post width 210 may also vary across the elevation dimension.

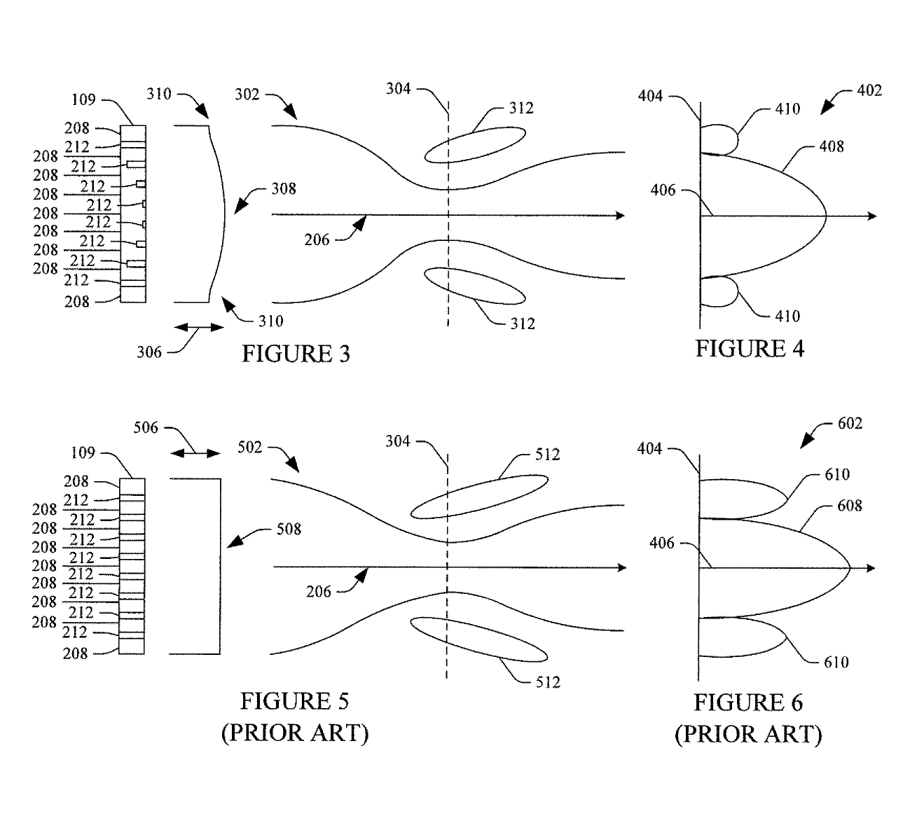

FIG. 3 illustrates an excitation energy distribution of the example transducer element 109 of FIG. 2 and a resulting beam profile 302 at a given focus depth 304, and FIG. 4 shows a magnitude profile 402 across the beam profile 302 at the focus depth 304.

In FIG. 3, the variable depth kerfs 212 produce an excitation profile 306 that rolls off from a central region 308 to end regions 310. As discussed herein, vibration, generally, is inversely proportional to kerf depth in that there is less vibration in portions of the element 109 in which the kerf depths are greater and vice versa. In the illustrated embodiment, kerfs depths are greater at the end regions 310, hence, the lower magnitude. FIG. 3 also shows out-of-plane energy 312 for the excitation profile 306. In FIG. 4, a y-axis 404 represents elevation and an x-axis 406 represents magnitude. The profile 402 includes a main lobe 408 and side lobes 410, which corresponds to the out-of-plane energy 312.

For comparative purposes, FIGS. 5 and 6 show a configuration of the transducer element in which the kerfs 212 have equal depth along the elevation direction and thus equal vibration, resulting in an excitation profile 506 with a constant magnitude 508. (A transducer element with no kerfs will also produce an excitation profile with a constant magnitude.) Likewise, a beam profile 502 is focused at the focus depth 304. In this embodiment, out-of-plane energy 512 is greater relative to the out-of-plane energy 312 of FIG. 3. As a result, as shown in FIG. 6, the profile 602 includes a main lobe 608 with a greater peak magnitude and a narrower width and side lobes 610 that are larger, relative to the main lobe 408 and side lobes 410 of FIG. 4.

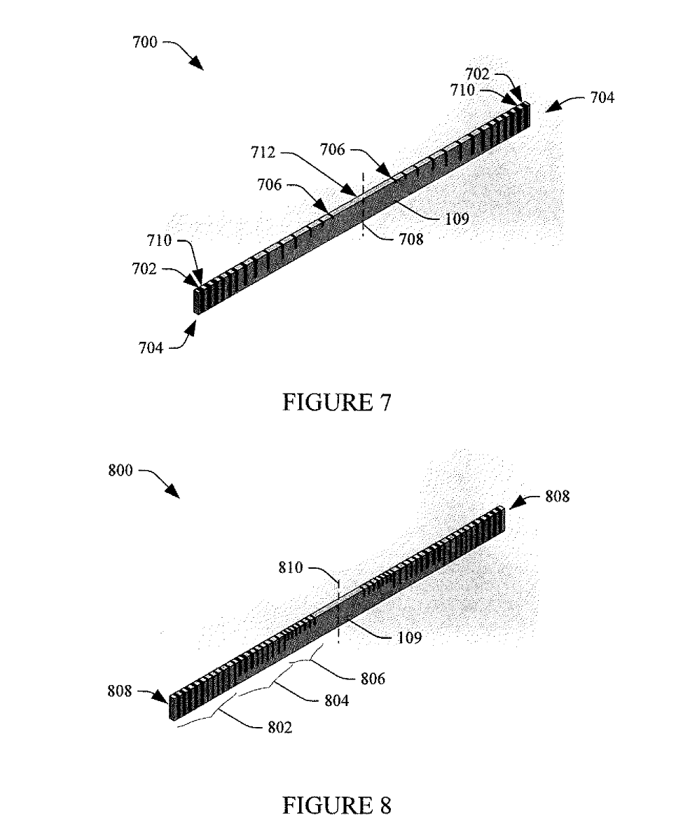

FIG. 7 illustrates perspective view 700 of another non-limiting example of the transducer element 109. In FIG. 7, kerfs vary in depth, as discussed herein. However, kerf widths also vary in size across elevation, from larger widths 702 at ends 704 of the element 109 and smaller widths 706 nearer a central region 708 of the element 109. Similar to FIG. 2, the kerf widths gradually decrease from the ends 704 to the central region 708. In this example, post widths also vary in size across elevation, however, from smaller widths 710 at the ends 704 of the element 109 to a largest width 712 at the central region 708 of the element 109. As a consequence of either or both, there is a decrease in the fractional amount of transducing material as the ends 704 relative the central region 708, and thus, further decrease in vibration at the ends 704.

FIG. 8 illustrates perspective view 800 of another non-limiting example of the transducer element 109. In FIG. 8, kerfs vary in depth, as discussed herein, except that depths of sets of kerfs 802, 804 and 806 decrease in discrete step-sizes from the ends 808 to the central region 810, such that the kerfs of the posts in set 802 are taller than the kerfs of the posts in set 806, . . . . Similar to FIG. 7, kerf widths also vary in size; however, in this example, kerfs widths vary across each of the sets of kerfs 802, 804 and 806. These kerf widths may vary in a continuous or discrete manner. Likewise, post widths vary in size across each of the sets of kerfs 802, 804 and 806. These post widths may also vary in a continuous or discrete manner. In a variation, the kerf widths and/or post widths may not vary across one or more of the sets.

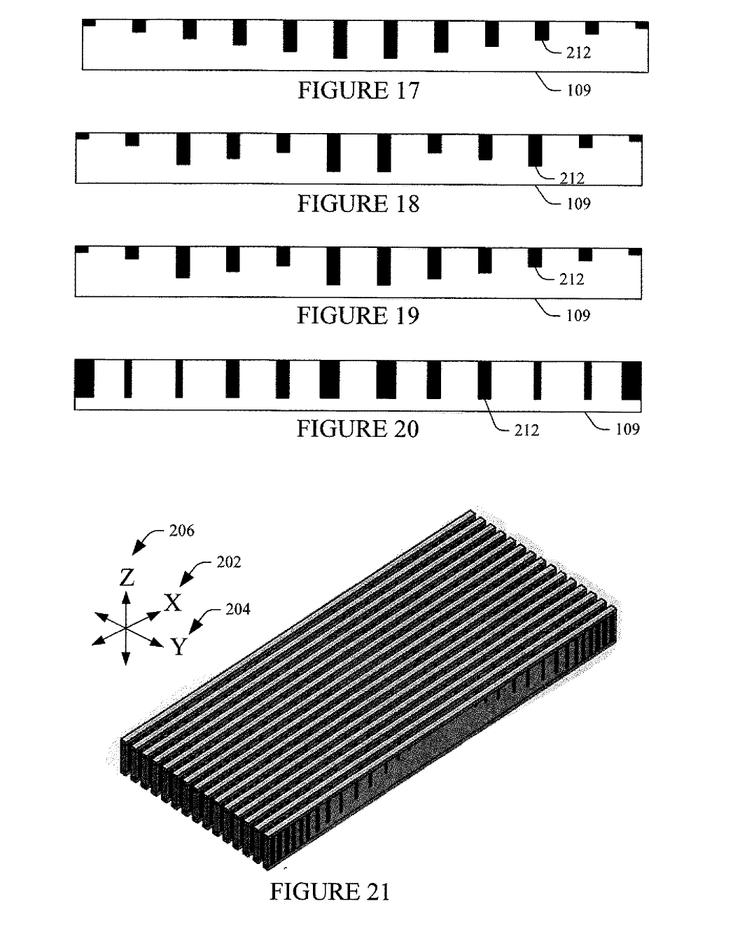

In FIG. 2, kerf depths decrease monotonically from the outer edges of the element 109 to the central region and are symmetric about the central region. In FIG. 17, the kerfs 212 have depths that increase monotonically from the outer edges of the element 109 to the central region and are symmetric about the central region. In FIG. 18, the kerfs 212 have depths that do not vary monotonically between the outer edges and the central region, but are symmetric about the central axis. In FIG. 19, the kerfs 212 have depths that do not vary monotonically between the outer edges and the central region and vary asymmetrically about the central axis. In FIGS. 17-19, kerf widths are equal and post widths are equal. However, at least two kerf widths may not be equal and/or at least two post widths may not be equal. In FIG. 20, kerf depths are equal and do not extend completely through the element 109, and neither kerf widths nor post widths are equal. In a variation, one or both the kerf widths or the post widths can be equal.

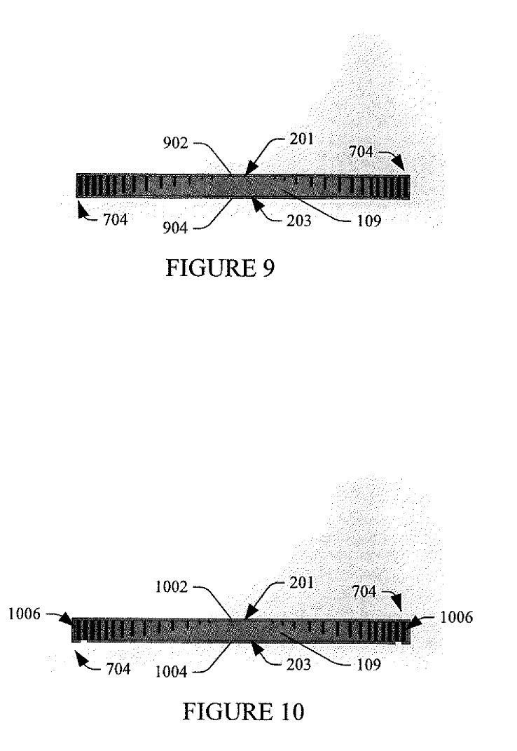

FIG. 9 illustrates the transducer element 109 with electrodes 902 and 904 affixed thereto. In this example, the electrode 902 is affixed to the surface 201 and extends between the ends 704, and the electrode 904 is affixed to the surface 203 and extends between the ends 704. With the configuration, the electrodes 902 and 904 are located on opposing sides of the element 109 and electrical connections thereto are on opposing sides of the element 109.

FIG. 10 illustrates another example of the transducer element 109 but with electrodes 1002 and 1004 affixed thereto. In this example, the electrode 1002 is affixed to the surface 201 and extends along sides 1006 of the element 109 and between the ends 704, whereas the electrode 1002 is affixed to the surface 203 and extends along a sub-portion of the element 109 between the ends 704. With the configuration, although the electrodes 1002 and 1004 are located on opposing sides of the element 109, electrical connections thereto can be on opposing sides or the same side of the element 109.

It is to be understood that the electrode configurations of FIGS. 9 and 10 are provided for explanatory purposes and are not limiting, and other approaches are also contemplated herein.

FIG. 11 shows the transducer element 109 of FIG. 9, with a backing layer or support 1102 affixed to the electrode 904 and a plurality of passive layers 1104 (two shown, but more or less can be included) affixed to the electrode 902. An acoustic lens (not shown) can be affixed to the plurality of passive layers 1104 to provide geometric focus. The plurality of passive layers 1104 and/or acoustic lens provides an impedance matching layer with skin of a subject being scanned. A gel or other fluid can be applied between the passive layers 1104 and the skin.

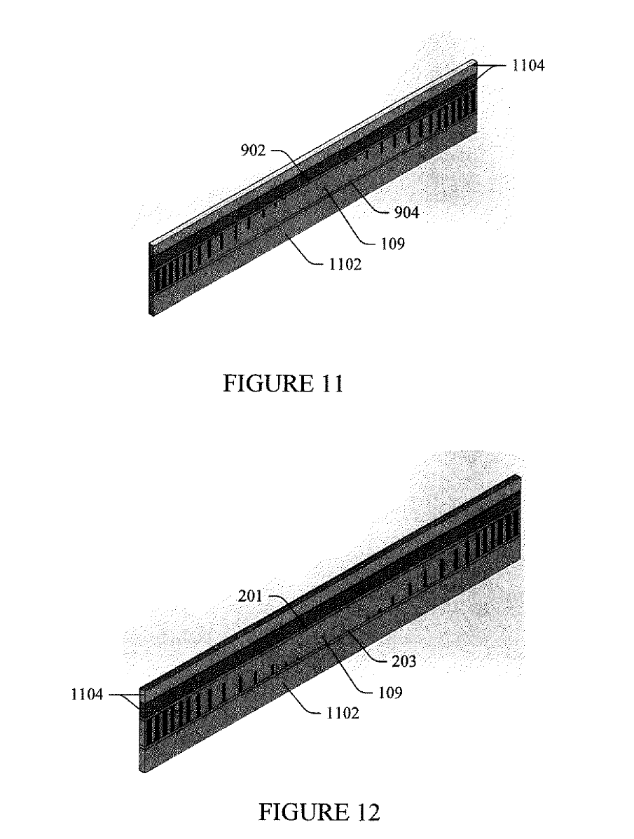

FIG. 12 is substantially similar to FIG. 11 except that the kerfs 212 extend from the surface 203 and in to element 109 instead of from the surface 201 and in to element 109. In yet another instance, a sub-set of the kerfs 212 extend from the surface 201 and into element 109 and another sub-set of the kerfs extend from the surface 203 and into the element 109.

Although the above examples include a rectangular prism shaped element 109, it is to be appreciated that the element 109 can be non-rectangular, for example, the surface 201 can be concave, convex, and/or otherwise shaped such that the element 109 is non-rectangular. In one or more of these configurations, even greater control over the spatial field characteristics can be accomplished. However, the rectangular embodiment may provide a more-easily-controlled manufacturing process.

FIG. 21 shows the element 109 of FIG. 9 in connection with a plurality of other elements 109 of FIG. 9 arranged in a one dimensional (1D) array. Again, reference numeral 204 represents the azimuth direction and reference numeral 202 represents the elevation direction. It is to be appreciated the elements 109 can also be arranged in a two dimensional (2D) array.

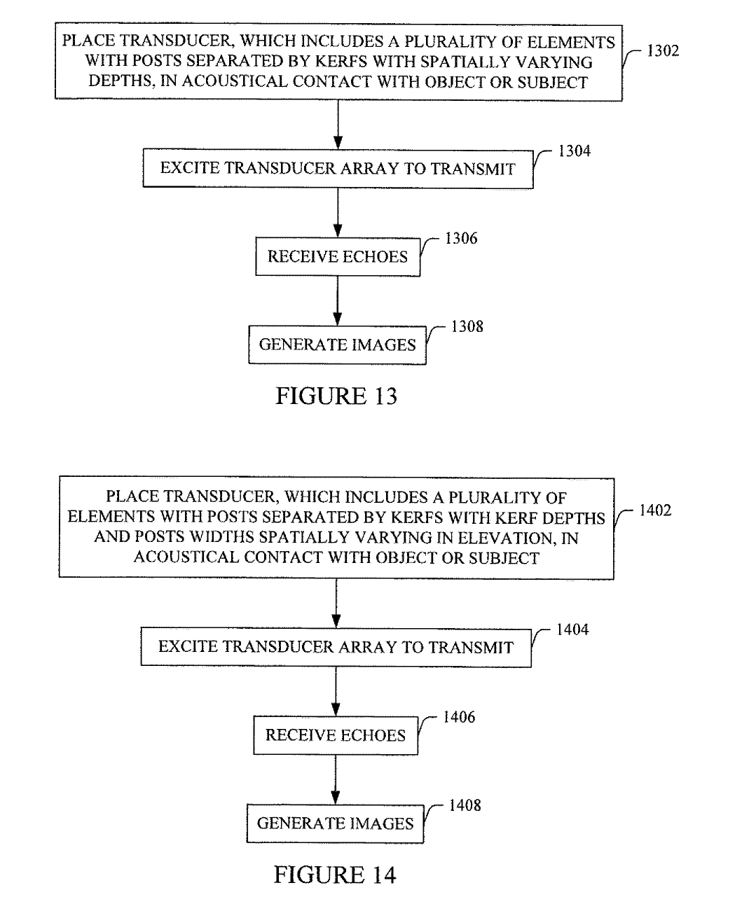

FIG. 13 illustrates a method in accordance with the imaging probe 102 described herein.

At 1302, a transducer array, which includes a plurality of elements 109 with transducing posts separated by non-transducing kerfs with depths that spatially vary (continuously or in discrete steps), is placed in acoustical contact with a subject or object.

At 1304, the transducer array is excited to transmit an ultrasound beam into the subject or object.

At 1306, the transducer array receives echoes produced in response to the ultrasound beam reflecting off structure in the subject or object.

At 1308, the echoes are processed to generate one or more images of the subject or object.

FIG. 14 illustrates another method in accordance with the imaging probe 102 described herein.

At 1402, a transducer array, which includes a plurality of elements 109 with transducing posts separated by non-transducing kerfs, wherein depths of the kerfs and widths of the posts spatially vary (continuously or in discrete steps), is placed in acoustical contact with a subject or object.

At 1404, the transducer array is excited to transmit an ultrasound beam into the subject or object.

At 1406, the transducer array receives echoes produced in response to the ultrasound beam reflecting off structure in the subject or object.

At 1408, the echoes are processed to generate one or more images of the subject or object.

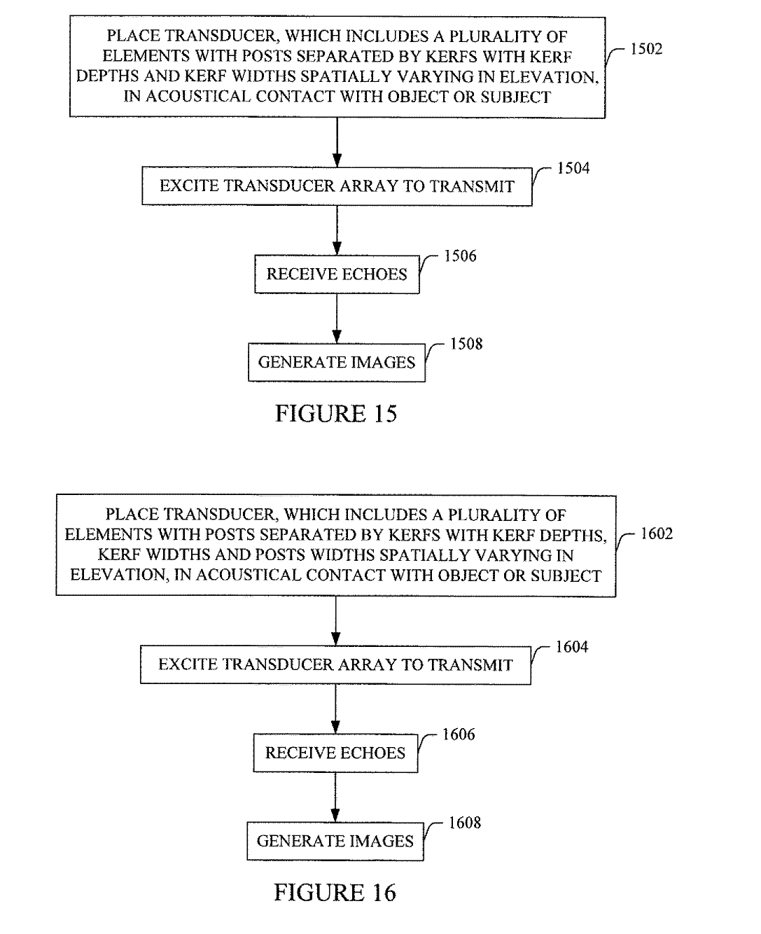

FIG. 15 illustrates another method in accordance with the imaging probe 102 described herein.

At 1502, a transducer array, which includes a plurality of elements 109 with transducing posts separated by non-transducing kerfs, wherein depths and widths of the kerfs spatially vary (continuously or in discrete steps), is placed in acoustical contact with a subject or object.

At 1504, the transducer array is excited to transmit an ultrasound beam into the subject or object.

At 1506, the transducer array receives echoes produced in response to the ultrasound beam reflecting off structure in the subject or object.

At 1508, the echoes are processed to generate one or more images of the subject or object.

FIG. 16 illustrates another method for in accordance with the imaging probe 102 described herein.

At 1602, a transducer array, which includes a plurality of elements 109 with transducing posts separated by non-transducing kerfs, wherein depths and widths of the kerfs and widths of the posts spatially vary (continuously or in discrete steps), is placed in acoustical contact with a subject or object.

At 1604, the transducer array is excited to transmit an ultrasound beam into the subject or object.

At 1606, the transducer array receives echoes produced in response to the ultrasound beam reflecting off structure in the subject or object.

At 1608, the echoes are processed to generate one or more images of the subject or object.

It is to be appreciated that the order of the above acts is provided for explanatory purposes and is not limiting. As such, one or more of the following acts may occur in a different order. Furthermore, one or more of the following acts may be omitted and/or one or more additional acts may be added.

In addition, the methods herein may be implemented by one or more processors executing computer executable instructions stored, encoded, embodied, etc. on computer readable storage medium such as computer memory, non-transitory storage, etc. In another instance, the computer executable instructions are additionally or alternatively stored in transitory or signal medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. An imaging probe, comprising:
a transducer array, with transducer elements with parallel first and second planar surface in which an ultrasound signal is emitted from the first planar surface, a transducer element, including: a plurality of transducing sub-elements arranged along an elevation direction in which adjacent transducing sub-elements are separated from leach other by kerfs non-transducing material, wherein depths of the kerfs vary along the elevation direction.

2. The imaging probe of claim 1, wherein a shape of the element is a rectangular prism.

3. The imaging probe of claim 1, wherein a depth of a kerf located nearer an end region of the array is greater than a depth of a kerf located nearer a central region of the array.

4. The imaging probe of claim 1, wherein the depths of the kerf vary symmetrically along the elevation direction.

5. The imaging probe of claim 1, wherein the depths of the kerf vary from kerf to kerf.

6. The imaging probe of claim 1, wherein the depths of the kerfs vary from sub-set to sub-set of kerfs, wherein a sub-set includes at least two kerfs having a same depth.

7. The imaging probe of claim 1, wherein the kerfs extend from the first planar surface into the array.

8. The imaging probe of claim 1, wherein the kerfs extend from the second planar surface into the array.

9. The imaging probe of claim 1, wherein a first sub-set of the kerfs extend from the first planar surface into the array and a second sub-set of the kerfs extend from the second planar surface into the array.

10. The imaging probe of claim 1, wherein the kerfs have a same width in the elevation direction and the transducing elements have a same width in the elevation direction.

11. The imaging probe of claim 1, wherein at least two of the kerfs have a different width in the elevation direction and the transducing sub-elements have a same width in the elevation direction.

12. The imaging probe of claim 1, wherein the kerfs have a same width in the elevation direction and at least two of the transducing sub-elements have different widths in the elevation direction.

13. The imaging probe of claim 1, wherein at least two of the kerfs have a different width in the elevation direction and at least two of the transducing sub-elements have different widths in the elevation direction.

14. A method, comprising:
exciting a transducer array, thereby producing an ultrasound beam that traverses an examination field of view, wherein the transducer array elements include a plurality of sub-elements arranged along an elevation direction wherein each element in elevation has kerfs of non-transducing material located between sub-elements, wherein depths of the kerfs vary along the elevation direction.

15. The method of claim 14, wherein the depths of the kerf vary from kerf to kerf.

16. The method 14, wherein the depths of the kerfs vary from sub-set to sub-set of kerfs, wherein a sub-set includes at least two kerfs having a same depth.

17. The method of claim 14, wherein at least two of the kerfs have a different width in the elevation direction.

18. The method of claim 14, wherein at least two of the transducing sub-elements have different widths in the elevation direction.

19. The method of claim 14, wherein an excitation profile of the ultrasound beam has at least one of a greater magnitude or a response at higher frequency near a central region of the array.

20. An ultrasound imaging system, comprising:
an imaging probe with transducer array elements that include sub-elements arranged along an elevation direction in which adjacent sub-elements are separated from each other by kerfs of non-transducing material with depths that extend only part way through the sub-elements; and
a console in electrical communication with the imaging probe, wherein the console controls transmission of an ultrasound signal by the array and processes echoes received by the array.

21. The ultrasound imaging system of claim 20, wherein the depths vary along the elevation direction.

22. The ultrasound imaging system of claim 21, wherein the depths of the kerfs vary monotonically from outer edges of a sub-element to a central region of the sub-element.

23. The ultrasound imaging system of claim 21, wherein the depths of kerfs nearer the outer edges are greater than the depths of the kerfs nearer the central region.

24. The ultrasound imaging system of claim 21, wherein the depths of kerfs nearer the central region are greater than the depths of the kerfs nearer the outer edges.

25. The ultrasound imaging system of claim 21, wherein the depths of the kerfs do not vary monotonically between outer edges of a sub-element to a central region of the sub-element.

26. The ultrasound imaging system of claim 20, wherein the depths vary symmetrically about the central region.

27. The ultrasound imaging system of claim 20, wherein the depths vary asymmetrically about the central region.

28. The ultrasound imaging system of claim 20, wherein the sub-elements are rectangular in shape.

29. The ultrasound imaging system of claim 20, wherein the sub-elements are not-rectangular in shape.
